(19) United States
(12) Patent Application Publication
(10) Pub. No.: US 2026/0139860 A1
(43) Pub. Date: May 21, 2026

(54) PRODUCTS OF MANUFACTURE FOR THE TREATMENT, PREVENTION AND AMELIORATION OF MICROBIAL INFECTIONS

(71) Applicant: Centre For Digestive Diseases, Five Dock (AU)

(72) Inventor: Thomas Julius Borody, Five Dock (AU)

(21) Appl. No.: 19/450,331

(22) Filed: Jan. 15, 2026

Related U.S. Application Data
(63) Continuation of application No. 17/239,570, filed on Apr. 24, 2021, now abandoned.
(60) Provisional application No. 63/014,945, filed on Apr. 24, 2020.

(51) Int. Cl. F24F 8/30; A61K 9/00; A61K 45/06; A61M 15/00; F24F 7/06

(52) U.S. Cl. CPC F24F 8/30; A61K 45/06; F24F 7/06; A61K 9/0075; A61M 15/002

(57) ABSTRACT

In alternative embodiments, provided are products of manufacture, including automobiles and buildings, and multiplexed systems such as ventilation systems, for treating, preventing, ameliorating, slowing the progress of, decreasing the severity of or preventing a microbial infection, such as bacterial and viral infections, including the common respiratory viruses such as influenza virus, respiratory syncytial virus, parainfluenza virus, adenovirus, rhinovirus, human metapneumovirus, hantaviruses, enterovirus, coronavirus (such as a SARS, MERS or COVID-19 infection), or an infection caused by a virus in the subfamily Orthocoronavirinae, or a virus in the family Coronaviridae, or a virus in the order Nidovirales.

### RELATED APPLICATIONS

This new United States Patent Application is a continuation of United States Patent Application Serial No. (USSN) Ser. No. 17/239,570, filed Apr. 24, 2021, now pending, which claims the benefit of priority under 35 U.S.C. § 1.19(e) to U.S. Provisional Patent Application Ser. No. 63/014,945 filed Apr. 24, 2020. The aforementioned applications are expressly incorporated herein by reference in their entirety and for all purposes.

### TECHNICAL FIELD

This invention generally relates to infectious diseases and medical devices. In alternative embodiments, provided are products of manufacture, including automobiles and buildings, and multiplexed systems such as ventilation systems, for treating, preventing, ameliorating, slowing the progress of, decreasing the severity of, or preventing a microbial infection, such as bacterial and viral infections, including the common respiratory viruses such as influenza virus, respiratory syncytial virus, parainfluenza virus, adenovirus, rhinovirus, human metapneumovirus, hantaviruses, enterovirus, coronavirus (such as a SARS, MERS or COVID-19 infection), or an infection caused by a virus in the subfamily Orthocoronavirinae, or a virus in the family Coronaviridae, or a virus in the order Nidovirales.

### BACKGROUND

Coronavirus infections have previously caused SARS (Severe Acute Respiratory Syndrome) and MERS (Middle East Respiratory Syndrome) and are particularly difficult to treat with anti-viral agents, and single drug regimens have not been found to be effective against the current coronavirus infection (2019-nCoV). The coronavirus infection (2019-nCoV), which started in China in December 2019, has spread rapidly throughout the world and has claimed hundreds of lives in China. The known coronavirus anti-infective agents used alone are unable to cure the infection.

### SUMMARY

In alternative embodiments, provided are products of manufacture for inactivating a microbe, optionally a bacteria or a virus,
wherein the product of manufacture is configured or manufactured to move air or force air under pressure, or create a positive air pressure flow of air, through a first duct, ductwork, vent, port, plenum (optionally an air delivery plenum and/or an air return plenum) and/or tube from an intake point, opening or value to an output point, opening or value,
and the product of manufacture comprises:
(a) a heating unit capable of or for heating the air (optionally forced air or positive air pressure flow of air) passing through the first duct, ductwork, vent, port, plenum and/or tube of the product of manufacture, wherein the heating unit is positioned at any point in the first duct, ductwork, vent, port, plenum and/or tube from the intake point, opening or value to the output point, opening or value,
and the heating unit is capable of heating the air to a temperature of between about 175° C. to about 500° C., or between about 200° C. to about 450° C., or between about 250° C. to about 400° C., and
a cooling unit positioned or operatively connected in or to the first duct, ductwork, vent, port, plenum and/or tube distal to the heating unit (between the heating unit and the output point, opening or value), and/or a cool or cold air intake duct, ductwork, vent, port, plenum, tube or value operatively connected to the first duct, ductwork, vent, port, plenum and/or tube distal to the heating unit, when cool or cold air is pumped or forced into the first duct, ductwork, vent, port, plenum and/or tube distal to the heating unit to lower the temperature of the air heated by the heating unit,
wherein optionally the cooling unit and/or the cool air intake duct, ductwork, vent, port, plenum, tube or value is configured, set or adjusted to lower the temperature of the heated air to between about to between about 50° C. to about 175° C., or between about 40° C. to about 150° C.,
and optionally the cooling unit comprises an air conditioning unit, optionally capable of delivering air having a temperature of between about 4° C. to about 40° C. to the first duct, ductwork, vent, port, plenum and/or tube distal to the heating unit;
(b) at least one or a plurality of air ionizers or ionizing devices capable of generating negative ionizations in an electric field in air (optionally forced air or positive air pressure flow of air) passing through the first duct, ductwork, vent, port, plenum and/or tube of the product of manufacture, wherein the at least one air ionizer or ionizing device is positioned at any point in the first duct, ductwork, vent, port, plenum and/or tube from the intake point, opening or value to the output point, opening or value,
and a positively charged collector plate coupled with or operatively connected to each of the at least one or plurality of air ionizers or ionizing devices, wherein the positively charged collector plate is positioned distal (between the at least one air ionizer or ionizing device and the output point, opening or value) to each of the at least one or plurality of air ionizers or ionizing devices,
and optionally the product of manufacture comprises two, three or four or more air ionizers or ionizing devices in the first duct, ductwork, vent, port, plenum and/or tube; or
(c) a combination of the heating device of (a) and the air ionizer or ionizing device of (b), wherein optionally the at least one air ionizer or ionizing device is not positioned between the heating device and the cooling device.

In alternative embodiments of products of manufacture as provided herein:
the product of manufacture is configured or manufactured as forced air or positive air pressure flow of air ventilation system and/or a central air system,
and optionally the air or forced air or positive air pressure flow of air is moved through the first duct, ductwork, vent, port, plenum and/or tube from the intake point, opening or value to the output point, opening or value by use of an air compressor,
and optionally the air compressor is or comprises: a positive displacement compressor (optionally a rotary or a reciprocating compressor), a gas compressor and/or a dynamic compressor (optionally a centrifugal or an axial compressor);
the product of manufacture is configured or manufactured as a HVAC (heating, venting, and air conditioning) system, optionally a forced air or positive air pressure flow of air HVAC system, or a dedicated outdoor air system (DOAS), and optionally the HVAC or the DOAS comprises a displacement ventilation (DV) system, a makeup air unit (MAU), a fresh air handling unit (FAHU), a packaged unit (PU) or a rooftop unit (RTU), or an air handling unit (AHU);
the product of manufacture is configured or manufactured as in or as part of a building, a room, an elevator, a subway, a tunnel, a vehicle, an automobile, a truck, an airplane, a boat, a bus, or a train,
and optionally the building is or comprises a house, an office building, a hospital, a waiting room, a bathroom, a theater, a store, a school or school room, an auditorium, a nursery, a restaurant, a hairdressing salon, a bar or a pub, or a shopping center;
the product of manufacture is configured or manufactured as a medical device,
and optionally the medical device is a respirator, an inhalation device, an asthma “puffer-like” device, an inhaler, a nebulizer or a nasal spray device,
and optionally the medical device is sleep apnea device, or a CPAP (continuous positive air pressure) device,
and optionally the medical device is a handheld device,
and optionally the inhalation device, asthma “puffer-like” device, inhaler, nebulizer or nasal spray device is a hand-held device, and optionally the inhaler, asthma “puffer-like” device, nebulizer or a nasal spray device is a metered or dose-counting inhaler, asthma “puffer-like” device or a nasal spray device,
and optionally the inhalation device, asthma “puffer-like” device, nebulizer or inhaler or the nasal spray device is manufactured or configured to fit over an individual's mouth and/or nose, or comprises a mouthpiece or facial mask, optionally a disposable mouthpiece or facial mask, that fits over the user's nose and/or mouth;
the product of manufacture is configured or manufactured as vacuum cleaner or a steam cleaner;
the product of manufacture further comprises at least one air filter disposed: in the first duct, ductwork, vent, port, plenum and/or tube at the intake point, opening or value; between the intake point, opening or value and the output point, opening or Value; between the heating unit and the output point, opening or value; and/or, at the output point, opening or value,
and optionally the at least one air filter is or comprises a high-efficiency particulate air (HEPA) filter or a high-efficiency particulate absorbing and high-efficiency particulate arrestance air filter, or an air filter capable of filtering out airborne viruses;
the heating unit that heats the air (optionally the forced air or positive air pressure flow of air) is a natural gas heater, a propane, heater, an oil heater, a coal heater, an electric heater, a solar heater and/or a heat pump;
the product of manufacture further comprises at least one thermostat operatively connected to:
the heating unit to adjust, control or set the temperature the air (optionally the forced air or positive air pressure flow of air) will be heated; and/or
the cooling unit, the first cool air intake tube, duct, ductwork, vent, port, plenum or value, and/or the additional cool air intake tube, duct, ductwork, vent, port, plenum or value to adjust, control or set the temperature the air (optionally the forced air or positive air pressure flow of air) will be cooled,
wherein optionally the at least one thermostat is operatively connected to a control panel or system, wherein optionally the control panel or system is remotely connected to the product of manufacture;
the product of manufacture further comprises: an air flow meter operatively connected to the air compressor; and or, a high-pressure cut-off switch operatively connected to the air compressor
the medical device, or the inhalation device, inhaler, asthma “puffer-like” device, nebulizer or nasal spray further comprises a humidifying or aerosol- or mist-delivering module or apparatus operatively connected to a tube or vent downstream of the cooling unit or is operatively connected to the first duct, ductwork, vent, port, plenum and/or tube distal to the heating unit, and the humidifying or aerosol- or mist-delivering module or apparatus is capable of delivering water moisture or an aerosol to the moved or forced air or positive air pressure flow of air,
and optionally the humidifying or aerosol- or mist-delivering module or apparatus delivers a medication or a drug, optionally by inhalation, to a user of the medical device,
and optionally the medication or the drug comprises an anti-microbial (optionally anti-viral or anti-microbial) medication or drug,
and optionally the medication or the drug is contained in a removable cassette or packette that can be inserted into a slot or port on the product of manufacture, wherein the removable cassette or packette is operatively connected to the first duct, ductwork, vent, port, plenum and/or tube distal to the heating unit, and the medication or the drug is delivered to or into the path of the airflow through the first duct, ductwork, vent, port, plenum and/or tube,
and optionally the connection of the removable cassette or packette to the first duct, ductwork, vent, port, plenum and/or tube comprises a value, optionally an adjustable value, that modulates or regulates the amount of the medication or the drug delivered to the to or into the path of the airflow through the first duct, ductwork, vent, port, plenum and/or tube,
and optionally the medication or drug comprises an antimicrobial agent or drug, a biofilm dissolving agent (optionally PULMOZYME™), an anticholinergic (optionally tiotropium bromide or SPIRIVA™), a bronchodilator, a corticosteroid, a antileukotriene, or an asthma drug (optionally formoterol, salbutamol, albuterol, or VENTOLIN™),
and optionally the anti-microbial agent or drug (optionally an anti-bacterial or an anti-viral or anti-microbial) medication or drug comprises:
an antibiotic, wherein optionally the antibiotic comprises gentimycin, or
lopinavir; ritonavir; oseltamivir (e.g., TAMIFLU™); lopinavir combined (formulated) with ritonavir, or KALETRA™, chloroquine phosphate, quinine, chloroquine diphosphate, hydroxychloroquine (e.g., PLAQUENIL™) or oral chloroquine (e.g., ARALEN™); remdesivir (e.g., GS-5734™, Gilead Sciences); nevirapine, efavirenz, emtricitabine, tenofovir (or the combination efavirenz with emtricitabine and tenofovir, or ATRIPLA™); amprenavir (e.g., AGENERASE™); nelfinavir (e.g., VIRACEPT™); a thiazolide class drug, optionally nitazoxanide (or ALINIA™, NIZONIDE™) or tizoxanide (or 2-Hydroxy-N-(5-nitro-2-thiazolyl)benzamide); plitidepsin (also known as dehydrodidemnin B), or APUDIN™ (PharmaMar, S.A.); an inhibitor or S-phase kinase-associated protein 2(SKP2 ), or dioscin, or niclosamide, or NICLOCIDE™, FENASAL™ or PHENASAL™; ribavirin; an interferon such as interferon alpha, interferon beta, interferon type I, interferon type II and/or interferon type III, or a combination of ribavirin and interferon beta, or a combination of lopinavir and ritonavir and interferon-beta-1b; abacavir, actemra, acyclovir e.g., (ACICLOVIR™), adefovir, amantadine, rintatolimod (e.g., AMPLIGEN™), amprenavir (e.g., AGENERASE™), aprepitant, arbidol, atazanavir, balavir, baloxavir marboxil (XOFLUZA™), bepotastine, bevirimat, bictegravir, biktarvy, brilacidin, cidofovir, caspofungin, lamivudine and zidovudine (e.g., COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, larnivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (e.g., BCX4430, IMMUCILLIN-A™), remdesivir (e.g., GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (e.g., RAPIVAB™), perfenazine, pleconaril, plurifioxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™) or zidovudine; a serine protease inhibitor, optionally camostat or camostat mesilate (or FOIPAN™); an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib: an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFCLIR™), troleandomycin (e.g. TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin; a macrocyclic lactone antibiotic, and optionally the macrocyclic lactone antibiotic comprises ivermectin (or STROMECTOL™, SOOLANTRA™); opaganib or YELIVA™; vitamin K; vitamin C; zinc; and/or aviptadil (or INVICORP™); or, any two, three, four, or five or more or combination thereof,
wherein optionally the combination comprises: opaganib or YELIVA™; azithromycin (or ZITHIROMAX™, or AZITHROCIN™) and hydroxychloroquine (e.g., PLAQUENIL™) or oral chloroquine (e.g., ARALEN™); or opaganib, azithromycin, hydroxychloroquine and a fourth drug or medication, optionally remdesivir, vitamin K; vitamin C; zinc; and/or aviptadil;
the product of manufacture is or is manufactured or fabricated as an automobile, a truck, a train, an airplane, a subway, or a boat, wherein the output point, opening or value opens into a passenger, pilot and/or operator compartment or compartments,
and optionally the cooling unit comprises an air conditioning unit or system, and optionally the cooling unit further comprises a cool or cold air intake duct, ductwork, vent, port, plenum, tube or value operatively connected to the first duct, ductwork, vent, port, plenum and/or tube distal to the heating unit,
and optionally the cool or cold air intake duct, ductwork, vent, port, plenum, tube or value comprises an intake port, opening or valve connected to the exterior of the automobile (or any vehicle) , truck, subway, train, airplane or boat for moving cool or cold air from the exterior to the first duct, ductwork, vent, port, plenum and/or tube distal to the heating unit;
the at least one or a plurality of air ionizers or ionizing devices operates at about 12 volts (V), or between about 10 and 15 V, and generates negative ionizations in an electric field, and optionally the at least one or a plurality of air ionizers or ionizing devices each can accelerate or generate a voltage of about 200,000 eV; and/or
the positively charged collector plate has a current of less than about 80μ amperes (μA).

In alternative embodiments, provided are methods for inactivating a microbe comprising use of a product of manufacture as provided herein, wherein optionally the microbe comprises a bacteria or a virus, and optionally the virus comprises a respiratory virus, and optionally the virus comprises an influenza virus, a respiratory syncytial virus, a parainfluenza virus, an adenovirus, a rhinovirus, a human metapneumovirus, a hantavirus, an enterovirus, a coronavirus (and optionally the coronavirus comprises a SARS, MERS or a COVID-19 infection causing virus), or a virus in the subfamily Orthocoronavirinae, or a virus in the family Coronaviridae, or a virus in the order Nidovirales.

In alternative embodiments, provided are use of a product of manufacture as provided herein for inactivating a microbe, wherein optionally the microbe comprises a bacteria or a virus, and optionally the virus comprises a respiratory virus, and optionally the virus comprises an influenza virus, a respiratory syncytial virus, a parainfluenza virus, an adenovirus, a rhinovirus, a human metapneumovirus, a hantavirus, an enterovirus, a coronavirus (and optionally the coronavirus comprises a SARS, MERS or a COVID-19 infection causing virus), or a virus in the subfamily Orthocoronavirinae, or a virus in the family Coronaviridae, or a virus in the order Nidovirales.

In alternative embodiments, provided are products of manufacture as provided herein for use in inactivating a microbe, wherein optionally the microbe comprises a bacteria or a virus, and optionally the virus comprises a respiratory virus, and optionally the virus comprises an influenza virus, a respiratory syncytial virus, a parainfluenza virus, an adenovirus, a rhinovirus, a human metapneumovirus, a hantavirus, an enterovirus, a coronavirus (and optionally the coronavirus comprises a SARS, MERS or a COVID-19 infection causing virus), or a virus in the subfamily Orthocoronavirinae, or a virus in the family Coronaviridae, or a virus in the order Nidovirales.

The details of one or more exemplary embodiments of the invention are set forth in the description below. Other features, objects, and advantages of the invention will be apparent from the description and from the claims.

All publications, patents, patent applications cited herein are hereby expressly incorporated by reference in their entireties for all purposes.

### DETAILED DESCRIPTION

In alternative embodiments, provided are products of manufacture, including vehicles of any type such as automobiles and trucks, and multiplexed systems such as ventilation systems, for treating, preventing, ameliorating, slowing the progress of, decreasing the severity of or preventing a microbial infection, such as bacterial and viral infections, including the common respiratory viruses such as influenza virus, respiratory syncytial virus, parainfluenza virus, adenovirus, rhinovirus, human metapneumovirus, hantaviruses, enterovirus, coronavirus (such as a SARS, MERS or COVID-19 infection), or an infection caused by a virus in the subfamily Orthocoronavirinae, or a virus in the family Coronaviridae, or a virus in the order Nidovirales.

In alternative embodiments as provided herein, products of manufacture process or sterilize air, for example, a flowing or forced air, e.g., a flowing or forced air, or positive air pressure flow of air, in a tube, vent, duct, ductwork, port, plenum or the like, to a very high temperature (a high heat) sufficient to kill or inactivate an airborne microbe such as a bacteria or a virus, after which the air is cooled and delivered via the tube, vent, duct, ductwork, port, plenum or the like, to a building space or compartment or a passenger space or compartment, e.g., on or in an any type of vehicle (e.g., an automobile, an ambulance, a bus, taxi, a truck, a military vehicle), a subway, a tunnel, an airplane, a boat, or a train.

For example, a product of manufacture as provided herein can be used to sterilize or inactivate airborne microbes in a building space or a passenger space or compartment (e.g., in a car (automobile) or a plane) by taking air from the space or compartment, sterilizing or inactivating the airborne microbes by the high heat treatment, and returning the sterilized or cleansed air back to the space or compartment. Alternatively, or additionally, air can be taken from outside the space or compartment, e.g., outside the building or vehicle, subway, tunnel, airplane, boat, bus or train, treated with the high heat to kill or inactivate any airborne microbes, and then delivered to inside the space or compartment.

In alternative embodiments, a product of manufacture as provided herein is configured or manufactured as in or as part of any type of or part of a structure or building, such as a room, a waiting room, a bathroom, an elevator. ln alternative embodiments, a product of manufacture as provided herein is configured or manufactured as in or as part of any type of vehicle (e.g., an automobile or car, a taxi, a truck, a military vehicle), a subway, a tunnel, an airplane, a boat, a bus, or a train. The building can be or can comprise a house, an office building, a hospital, a theater, a store, a school or school room, an auditorium, a nursery, a restaurant, a hairdressing salon, a bar or a pub, or a shopping center.

In alternative embodiments, a drug delivery device as provided herein, for example, the inhaler, asthma “puffer-like” device, nebulizer, or the nasal spray device is, or comprises parts of or is made or used, by a method or a device as described in e.g., U.S. Pat. Nos. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol or aerosol imparted with flavor, a sensor configured to detect a predefined variable, an interface configured to make a notification to an inhaler of the aerosol, and a controller), or U.S. Pat. No. 10,463,815 (describing a dry powder inhaler may include a powder storage region, an inlet channel, a dispersion chamber, and an outlet channel); or U S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol and/or a vapour in an inhaler device includes a reservoir for storing a supply of a liquid; a heating system fluidly connected with the reservoir for receiving the liquid and configured to heat the liquid to generate the aerosol and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, e.g., as described in U.S. Pat. No. 10,561,808.

### Heating Units

In alternative embodiments, products of manufacture comprise a heating unit capable of or for heating the air (optionally forced air or positive air pressure flow of air) passing through the first duct, ductwork, vent, port, plenum and/or tube of the product of manufacture from the intake point, opening or value to the output point, opening or value to a temperature sufficient to kill or inactivate an airborne microbe such as a respiratory virus such as COVID-19, a rhinovirus or influenza. In alternative embodiments, the heating unit heats the air to a temperature of between about 175° C. to about 500° C., or between about 200° C. to about 450° C., or between about 250° C. to about 400° C., and optionally the exact temperature can be preset or can be adjusted or adjustable, and optionally a thermostat is operably linked to the heating unit to ensure a preset air temperature or an air temperature set by an adjustable control is attained.

In alternative embodiments, the heating unit comprises heating wires or coils, or is gas or fuel (flame) heated air. In alternative embodiments, the heating wires or coils comprise or comprise use of resistance coil heating elements, or resistance wire open coil heating elements, which can be wire or ribbon, straight or coiled. In alternative embodiments, the heating wires or coils comprise or comprise use of nichrome, e.g., a nichrome 80/20(80 % nickel, 20% chromium) wire, ribbon, or strip; or, iron-chromium-aluminium (FeCrAl) alloys such as KANTHAL™.

In alternative embodiments, if the product of manufacture is or is part of vehicle such as an automobile (a car), a truck, or the like, the heating unit comprises use of the vehicles liquid or natural gas reservoir (e.g., from a gas tank, e.g., fuel used to power the car) to generate a flame to heat the air.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12%, 11%, 10%, 9%, 8%, 7%, 6%, 5%, 4%, 3%, 2%, l%, 0.5%, 0.1%, 0.05%, or 0.01% of the stated value. Unless otherwise clear

from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

What is claimed is:

1. A product of manufacture for inactivating a microbe, optionally a bacteria or a virus,
wherein the product of manufacture is configured or manufactured to move air or force air under pressure, or create a positive air pressure flow of air, through a first duct, ductwork, vent, port, plenum (optionally an air delivery plenum and/or an air return plenum) and/or tube from an intake point, opening or value to an output point, opening or value,
and the product of manufacture comprises:
(a) a heating unit capable of or for heating the air (optionally forced air or positive air pressure flow of air) passing through the first duct, ductwork, vent, port, plenum and/or tube of the product of manufacture, wherein the heating unit is positioned at any point in the first duct, ductwork, vent, port, plenum and/or tube from the intake point, opening or value to the output point, opening or value,
and the heating unit is capable of heating the air to a temperature of between about 175° C. to about 500° C., or between about 200° C. to about 450° C., or between about 250° C. to about 400° C., and
a cooling unit operatively connected to the first duct, ductwork, vent, port, plenum and/or tube distal to the heating unit (between the heating unit and the output point, opening or value), and/or a cool or cold air intake duct, ductwork, vent, port, plenum, tube or value operatively connected to the first duct, ductwork, vent, port, plenum and/or tube distal to the heating unit, when cool or cold air is pumped or forced into the first duct, ductwork, vent, port, plenum and/or tube distal to the heating unit to lower the temperature of the air heated by the heating unit,
wherein optionally the cooling unit and/or the cool air intake duct, ductwork, vent, port, plenum, tube or value is configured, set or adjusted to lower the temperature of the heated air to between about to between about 50° C. to about 175° C., or between about 40° C. to about 150° C.,
and optionally the cooling unit comprises an air conditioning unit, optionally capable of delivering air having a temperature of between about 4° C. to about 40° C. to the first duct, ductwork, vent, port, plenum and/or tube distal to the heating unit
(b) at least one or a plurality of air ionizers or ionizing devices capable of generating negative ionizations in an electric field in air (optionally forced air or positive air pressure flow of air) passing through the first duct, ductwork, vent, port, plenum and/or tube of the product of manufacture, wherein the at least one air ionizer or ionizing device is positioned at any point in the first duct, ductwork, vent, port, plenum and/or tube from the intake point, opening or value to the output point, opening or value,
and a positively charged collector plate coupled with or operatively connected to each of the at least one or plurality of air ionizers or ionizing devices, wherein the positively charged collector plate is positioned distal (between the at least one air ionizer or ionizing device and the output point, opening or value) to each of the at least one or plurality of air ionizers or ionizing devices,
and optionally the product of manufacture comprises two, three or four or more air ionizers or ionizing devices in the first duct, ductwork, vent, port, plenum and/or tube, or
(c) a combination of the heating device of (a) and the air ionizer or ionizing device of (b), wherein optionally the at least one air ionizer or ionizing device is not positioned between the heating device and the cooling device.

2. The product of manufacture of claim 1, wherein the product of manufacture is configured or manufactured as forced air or positive air pressure flow of air ventilation system and/or a central air system,
and optionally the air or forced air or positive air pressure flow of air is moved through the first duct, ductwork, vent, port, plenum and/or tube from the intake point, opening or value to the output point, opening or value by use of an air compressor,
and optionally the air compressor is or comprises: a positive displacement compressor (optionally a rotary or a reciprocating compressor), a gas compressor and/or a dynamic compressor (optionally a centrifugal or an axial compressor).

3. The product of manufacture of claim 1, wherein the product of manufacture is configured or manufactured as a HVAC (heating, venting, and air conditioning) system, optionally a forced air or positive air pressure flow of air HVAC system, or a dedicated outdoor air system (DOAS), and optionally the HVAC or the DOAS comprises a displacement ventilation (DV) system, a makeup air unit (MAU), a fresh air handling unit (FAHU), a packaged unit (PU) or a rooftop unit (RTU), or an air handling unit (AHU).

4. The product of manufacture of claim 1, wherein the product of manufacture is configured or manufactured as in or as part of a building, a room, an elevator, a subway, a tunnel, a vehicle, an automobile, a truck, an airplane, a boat, a bus, or a train,
and optionally the building is or comprises a house, an office building, a hospital, a waiting room, a bathroom, a theater, a store, a school or school room, an auditorium, a nursery, a restaurant, a hairdressing salon, a bar or a pub, or a shopping center.

5. The product of manufacture of claim 1, wherein the product of manufacture is configured or manufactured as a medical device,
and optionally the medical device is a respirator, an inhalation device, an asthma “puffer-like” device, nebulizer, an inhaler, a nebulizer or a nasal spray device,
and optionally the medical device is sleep apnea device, or a CPAP (continuous positive air pressure) device,
and optionally the medical device is a handheld device,
and optionally the inhalation device, inhaler, asthma “puffer-like” device, nebulizer or nasal spray device is a hand-held inhaler, asthma “puffer-like” device, nebulizer or nasal spray device, and optionally the inhaler asthma “puffer-like” device, nebulizer or nasal spray device is a metered or dose-counting inhaler, asthma “puffer-like” device, nebulizer or nasal spray device,
and optionally the inhalation device, asthma “puffer-like” device, nebulizer, inhaler or nasal spray device is manufactured or configured to fit over an individual's mouth and/or nose.

6. The product of manufacture of claim 1, wherein the product of manufacture is configured or manufactured as vacuum cleaner or a steam cleaner.

7. The product of manufacture of claim 1, further comprising at least one air filter disposed: in the first duct, ductwork, vent, port, plenum and/or tube at the intake point, opening or value; between the intake point, opening or value and the output point, opening or value; between the heating unit and the output point, opening or value; and/or, at the output point, opening or value,
and optionally the at least one air filter is or comprises a high-efficiency particulate air (HEPA) filter or a high-efficiency particulate absorbing and high-efficiency particulate arrestance air filter, or an air filter capable of filtering out airborne viruses.

8. The product of manufacture of claim 1, wherein the heating unit that heats the air (optionally the forced air or positive air pressure flow of air) is a natural gas heater, a propane, heater, an oil heater, a coal heater, an electric heater, a solar heater and/or a heat pump.

9. The product of manufacture of claim 1, further comprising at least one thermostat operatively connected to:
the heating unit to adjust, control or set the temperature the air (optionally the forced air or positive air pressure flow of air) will be heated; and/or
the cooling unit, the first cool air intake tube, duct, ductwork, vent, port, plenum or value, and/or the additional cool air intake tube, duct, ductwork, vent, port, plenum or value to adjust, control or set the temperature the air (optionally the forced air or positive air pressure flow of air) will be cooled,
wherein optionally the at least one thermostat is operatively connected to a control panel or system, wherein optionally the control panel or system is remotely connected to the product of manufacture.

10. The product of manufacture of claim 1, further comprising: an air flow meter operatively connected to the air compressor; and or, a high-pressure cut-off switch operatively connected to the air compressor.

11. The product of manufacture of claim 5, wherein the medical device, or the inhalation device, asthma “puffer-like” device, inhaler, nebulizer, or nasal spray further comprises a humidifying or aerosol- or mist-delivering module or apparatus operatively connected to a tube or vent downstream of the cooling unit or is operatively connected to the first duct, ductwork, vent, port, plenum and/or tube distal to the heating unit, and the humidifying or aerosol- or mist-delivering module or apparatus is capable of delivering water moisture or an aerosol to the moved or forced air or positive air pressure flow of air,
and optionally the humidifying or aerosol- or mist-delivering module or apparatus delivers a medication or a drug, optionally by inhalation, to a user of the medical device,
and optionally the medication or the drug comprises an anti-microbial (optionally anti-viral or anti-microbial) medication or drug,
and optionally the medication or the drug is contained in and delivered via a reservoir (optionally a refillable reservoir) in or on the product of manufacture, or a removable cassette or packette that can be inserted into a slot or port on the product of manufacture, or a separate reservoir or container operatively linked or joined to the product of manufacture, wherein the reservoir, container or removable cassette or packette is operatively connected to the first duct, ductwork, vent, port, plenum and/or tube distal to the heating unit, and the medication or the drug is delivered to or into the path of the airflow through the first duct, ductwork, vent, port, plenum and/or tube,
and optionally the connection of the removable cassette or packette to the first duct, ductwork, vent, port, plenum and/or tube comprises a value, optionally an adjustable value, that modulates or regulates the amount of the medication or the drug delivered to the to or into the path of the airflow through the first duct, ductwork, vent, port, plenum and/or tube,
and optionally the medication or drug comprises an antimicrobial agent or drug, a biofilm dissolving agent, an anticholinergic (optionally tiotropium, bromide or SPIRIVA™), a bronchodilator, a corticosteroid, a antileukotriene, or an asthma drug (optionally formoteml, salbutamol, albuterol, or VENTOLIN™),
and optionally the anti-microbial agent or drug (optionally an anti-bacterial or an anti-viral or anti-microbial) medication or drug comprises:
an antibiotic, wherein optionally the antibiotic comprises gentimycin, or
and optionally the medication or drug comprises lopinavir; ritonavir; oseltarnivir (e.g., TAMIFLU™); lopinavir combined (formulated) with ritonavir, or KALETRA™; chloroquine phosphate, quinine, chloroquine diphosphate, hydroxychloroquine (e.g., PLAQUENIL™) or oral chloroquine (e.g., ARALEN™);
remdesivir (e.g., GS-5734™, Gilead Sciences); nevirapine, efavirenz, emtricitabine, tenofovir (or the combination efavirenz with emtricitabine and tenofovir, or ATRIPLA™); amprenavir (e.g., AGENERASE™); nelfinavir (e.g., VIRACEPT™); a thiazolide class drug, optionally nitazoxanide (or ALINIA™, NIZONIDE™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2(SKP2 ), or dioscin, or niclosamide, or NICLOCIDE™, FENASAL™, or PHENASAL™; ribavirin; an interferon such as interferon alpha, interferon beta, interferon type I, interferon type II and/or interferon type III, or a combination of ribavirin and interferon beta, or a combination of lopinavir and ritonavir and interferon-beta-lb; abacavir, actemra, acyclovir e.g, (ACICLOVIR™), adefovir, amantadine, rintatolimod (e.g., AMPLIGEN™), amprenavir (e.g., AGENERASE™), aprepitant, arbidol, atazanavir, balavir, baloxavir marboxil (XOFLUZA™), bepotastine, bevirimat, bictegravir, biktarvy, brilacidin, cidofovir, caspofungin, lamivudine and zidovudine (e.g., COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (e.g., BCX4430, IMMUCILLIN-A™), remdesivir (e.g., GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (e.g., RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™) or zidovudine; a serine protease inhibitor, optionally camostat or camostat mesilate (or FOIPAN™); an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin; a macrocyclic lactone antibiotic, and optionally the macrocyclic lactone antibiotic comprises ivermectin (or STROMECTOL™, SOOLANTRA™); opaganib or YELIVA™; or, any two, three, four or five or more or combination thereof:
wherein optionally the combination comprises: opaganib or YELIVA™; azithromycin (or ZITHROMAX™, or AZITHROCIN™) and hydroxychloroquine (e.g., PLAQUENIL™) or oral chloroquine (e.g., ARALEN™); or opaganib, azithromycin, hydroxychloroquine and a fourth drug, optionally remdesivir.

12. The product of manufacture of claim 4, wherein the product of manufacture is or is manufactured or fabricated as an automobile, a truck, a train, an airplane, a subway, or a boat, wherein the output point, opening or value opens into a passenger, pilot and/or operator compartment or compartments,
and optionally the cooling unit comprises an air conditioning unit or system, and optionally the cooling unit further comprises a cool or cold air intake duct, ductwork, vent, port, plenum, tube or value operatively connected to the first duct, ductwork, vent, port, plenum and/or tube distal to the heating unit,
and optionally the cool or cold air intake duct, ductwork, vent, port, plenum, tube or value comprises an intake port, opening or valve connected to the exterior of the automobile, truck, subway, train, airplane or boat for moving cool or cold air from the exterior to the first duct, ductwork, vent, port, plenum and/or tube distal to the heating unit.

13. The product of manufacture of claim 1, wherein the at least one or a plurality of air ionizers or ionizing devices operates at about 12 volts (V), or between about 10 and 15 V, and generates negative ionizations in an electric field, and optionally the at least one or a plurality of air ionizers or ionizing devices each can accelerate or generate a voltage of about 200,000 eV.

14. The product of manufacture of claim 1, wherein the positively charged collector plate has a current of less than about 80μamperes (μA).

15. The product of manufacture of claim 5, wherein the medical device, or the inhalation device, asthma “puffer-like” device, inhaler, nebulizer, or nasal spray further comprises a humidifying or aerosol- or mist-delivering module or apparatus operatively connected to a tube or vent downstream of the cooling unit or is operatively connected to the first duct, ductwork, vent, port, plenum and/or tube distal to the heating unit, and the humidifying or aerosol- or mist-delivering module or apparatus is capable of delivering water moisture or an aerosol to the moved or forced air or positive air pressure flow of air,
and optionally the humidifying or aerosol- or mist-delivering module or apparatus delivers a medication or a drug, optionally by inhalation, to a user of the medical device,
and optionally the medication or the drug comprises:
(a) opaganib, or YELIVA™, or opaganib, or YELIVA™ and oral and/or inhaled or aerosol chloroquine (or ARALEN™), chloroquine phosphate, chloroquine diphosphate and/or hydroxychloroquine (optionally, PLAQUENIL™),
and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc (optionally zinc sulfate, optionally at (50 mg daily);
(b) lopinavir, ritonavir and oseltamivir (optionally, TAMIFLU™), and/or zanamivir (or RELENZA™);
(c) lopinavir combined (formulated) with ritonavir, or KALETRA™, ALTERA™, ALUVIA™, KALNIELTREX, LOPIMUNE™ or LOPINAVIR™, and/or zanamivir (or RELENZA™), wherein optionally lopinavir and ritonavir separately formulated;
(d) lopinavir combined (formulated) with ritonavir (or KALETRA™, ALTERA™, ALUVIA™, KALMELTREX, LOPIMUNE™ or LOPINAVIR™), or lopinavir and ritonavir, and oseltamivir (optionally, TAMIFLU™), and/or zanamivir (or RELENZA™), and optionally also with inhaled or aerosol formulations or versions of chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously;
(e) lopinavir, ritonavir, chloroquine and oseltamivir (or TAMIFLU™); wherein optionally the chloroquine comprises inhaled or aerosol chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously;
(f) lopinavir and oseltamivir (optionally, TAMIFLU™), and/or zanamivir (or RELENZA™);
(g) ritonavir and oseltamivir (optionally, TAMIFLU™), and/or zanamivir (or RELENZA™);
(h) remdesivir (optionally, GS-5734™, Gilead Sciences) alone, or oseltamivir (optionally, TAMIFLU™) and remdesivir (optionally, GS-5734™, Gilead Sciences), and optionally the remdesivir is an oral formulation and/or an inhaled or aerosol remdesivir formulation;
(i) oseltamivir (optionally, TAMIFLU™) and efavirenz (optionally, SUSTIVA™), molnupiravir and/or zanamivir (or RELENZA™);
(j) oseltamivir (optionally, TAMIFLU™) and nevirapine (or the combination efavirenz or molnupiravir with emtricitabine and tenofovir, or ATRIPLA™);
(k) oseltamivir (or TAMIFLU™) and amprenavir (optionally, AGENERASE™);
(l) oseltamivir (optionally, TAMIFLU™) and nelfinavir (optionally, VIRACEPT™); or
(m) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (l), or any drug or drug combination as provided herein, optionally a thiazolide class drug, optionally nitazoxanide, with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, or the thiazolide class drug (optionally, nitazoxanide or tizoxanide) and oseltamivir (or TAMIFLU™), and optionally the thiazolide class drug (optionally, nitazoxanide or tizoxanide) is formulated or administered with ribavirin or tribavirin (or COPEGUS™, REBETO™, or VIRAZOLE™), and an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and optionally formulated and administered as an inhalant or a mist (optionally using a nebulizer, nasal spray or equivalent);
(n) plitidepsin (also known as dehydrodidemnin B), or APLIDIN™ (PharmaMar, S.A.);
(o) an inhibitor or S-phase kinase-associated protein 2(SKP2 ), or dioscin, or nidosamide, or NICLOCIDE™, FENASAL™ or PHENASAL™;
(p) ribavirin or tribavirin (or COPEGUS™ REBETOL™ or VIRAZOLE™), interferon beta 1b, or a combination of ribavirin and interferon beta, or a combination of lopinavir and ritonavir and interferon-beta-1b;
(q) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-A™), remdesivir (optionally, GS-5734™, Gilead Sciences), cytarabine, gemcitabine, emtricitabine, lamivudine, zalcitabine, abacavir, acyclovir, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir, saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
(r) a mucolytic therapy or drug, optionally acetylcysteine, ambroxol, bromhexine, carbocisteine, erdosteine, mecysteine or dornase alfa, or an expectorant, optionally guaifenesin;
(s) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat;
(t) an angiotensin-converting enzyme 2(ACE 2 ) inhibitor, optionally to block the site of viral spike protein interaction for anti-SARS-CoV-2 infection control;
(u) an anti-vascular endothelial growth factor (VEGF) (optionally VEGF-A) drug or antibody, optionally bevacizumab;
(v) a protease inhibitor, optionally danoprevir, optionally a serine protease inhibitor, optionally camostat or narlaprevir (optionally ARLANSA™);
(w) anti-PD-1 checkpoint inhibitor, optionally camrelizumab;
(x) a compound or antibody capable of binding complement factor C5 and blocking membrane attack complex formation, optionally eculizumab;
(y) a cathepsin inhibitor, optionally a cathepsin K, B or L inhibitor, optionally relacatib;
(z) thalidomide, or thalidomide and glucocorticoid (optionally low-dose glucocorticoid), or and thalidomide and celecoxib;
(aa) an antibacterial antibiotic or a macrolide drug,
wherein optionally the macrolide drug comprises azithromycin, optionally dosaged at between about 50 mg to about 2000 mg per dose or per day (optionally, ZITHROMAX™, or AZITHROCIN™, optionally an oral extended-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,
wherein optionally the antibacterial antibiotic or the macrolide drug. optionally azithromycin, is formulated for normal release or as a delayed release formulation,
and optionally the antibacterial antibiotic comprises a tetracycline class drug, a glycylcycline or a fluorocycline class drug, or an analogue thereof, and optionally the tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™ CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof,
and optionally the antibacterial, optionally azithromycin, optionally dosaged at between about 50 mg to about 2000 mg per dose or per day (optionally, ZITHROMAX™, or AZITHROCIN™, optionally an oral extended-release formulation of azithromycin, or ZMAX™), is administered with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or cholecalciferol (vitamin D3) or calcifediol,
and optionally the antibacterial antibiotic comprises an antimycobacterial drug, and optionally the antimycobacterial drug comprises clofazimine (optionally LAMPRENE™);
(bb) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 240 mg per day,
optionally formulated or administered with an antibiotic (optionally azithromycin, minocydine, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder,
and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), is formulated with and/or administered with chloroquine (or ARALEN™), chloroquine phosphate, chloroquine diphosphate and/or hydroxychloroquine (optionally, PLAQUENIL™) with or without zinc, and optionally this combination is administered weekly, or every two week, or one every 5 to 28 days, as a prophylactic,
and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,
and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), is administered alone for 1, 2, 3, 4, 5, 6, 7, 8, 9, 10 days or up to 20 or more days, followed by administration of an antibiotic (optionally doxycycline) for a corresponding period of days, and optionally repeating the cycle of dosaging,
and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), is formulated or administered with:
(i) at least one antibiotic (wherein optionally the antibiotic is doxycycline (optionally, DORYX™, DOXYHEXA™, DOXYLIN™) (optionally formulated or administered at a dosage of between about 25 mg to 500 mg), or azithromycin, optionally dosaged at between about 50 mg to about 2000 mg per dose or per day (optionally, ZITHROMAX™, or AZITHROCIN™, optionally an oral extended-release formulation of azithromycin, or ZMAX™) (optionally formulated or administered at a dosage of between an about 50 mg to 2000 mg);
(ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
(iii) a zinc (optionally a zinc sulphate, acetate, gluconate or picolinate) optionally formulated or administered at a dosage of between about 1 mg to 250 mg; and/or
(iv) at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day,
and optionally the ivermectin (optionally STROMECTOL™), moxidectin optionally CYDECTIN™, EQUEST™, QUEST™), selamectin (optionally STRONGHOLD™), milbemycin (optionally milbemectin, milbemycin oxime, moxidectin or nemadectin) or doramectin (optionally DECTOMAX™) is administered or formulated alone or in combination with any of the above (i) to (iv) (for example, at least one antibiotic, chloroquine (or ARALEN™), chloroquine phosphate, chloroquine diphosphate and/or hydroxychloroquine (optionally, PLAQUENIL™), zinc and/or at least one vitamin are formulated (and administered) as oral formulations (for example, as tablets, capsules, gels or gel tabs), injectable formulations, powders (for example, for inhalation or for addition to an ingestible liquid) or liquids (for example, for ingestion, infusion or injection);
(cc) chloroquine (or ARALEN™), chloroquine phosphate, chloroquine diphosphate and/or hydroxychloroquine (optionally, PLAQUENIL™) with (or formulated with) or in combination with any of (a) to (bb), or chloroquine, chloroquine phosphate, chloroquine diphosphate and/or hydroxychloroquine (optionally, PLAQUENIL™) and oseltamivir (or TAMIFLU™);
(dd) chloroquine (optionally, ARALEN™), chloroquine phosphate, chloroquine diphosphate and/or hydroxychloroquine (optionally, PLAQUENIL™) alone or with:
(i) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), optionally at a dosage of between about 10 mg to 80 mg dosages, or 5, 6, 7, 8, 9, 10, 11 or 12 to 60 mg dosages, and/or
(ii) vitamin D, vitamin D2 (or ergocalciferol), vitamin D3 (or cholecalciferol) or calcifediol, optionally at a dosage of between about 3,000 to 100,000 units per day, or between about 10,000 to 50,000 units a day, or
(iii) with (i) and (ii) and zinc (optionally a zinc sulphate, acetate, gluconate or picolinate) optionally at a dosage of between about 1 mg to 250 mg, or
(iv) the combination of (iii) also with a tetracycline class drug, wherein optionally the tetracycline class drug comprises doxycycline, or DORYX™, DOXYHEXA™, DOXYLIN™, optionally dosages at between about 25 mg to 600 mg per day, optionally between about 100 mg to 500 mg or between about 200 mg to 400 mg per day or per dose;
(ee) colchicine, or COLCRYS™, MITIGARE™, optionally administered or dosaged at between about 0.5 mg to 20 mg, or about 1 mg to 15 mg, or about 3 mg to mg, or about 4 mg to 6 mg, per day for a period of between about 7 and 21 days, or about 14 days, and optionally also administered or formulated with an antibiotic (optionally azithromycin or doxycycline), ivermectin, hydroxychloroquine (optionally, PLAQUENIL™) and/or zinc (optionally zinc sulfate, optionally at (50 mg daily);
(ff) a corticosteroid class drug such as budesonide (optionally RHINOCORT™ or PULMICORT™), prednisolone (or ORAPRED™), methyl-prednisolone, prednisone (or DELTASONE™ or ORASONE™) or hydrocortisone (or CORTEF™),
and optionally the corticosteroid class drug (for example budesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
and optionally the corticosteroid class drug is formulated as a powder or for administration in an inhaler or by nasal spray, or for rectal administration, and optionally the budesonide is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbernectin, milbemycin oxime, moxidectin or nemadectin), doramectin (optionally DECTOMAX™), an antibiotic (optionally azithromycin or a tetracycline class drug, and optionally the tetracycline class drug comprises doxycycline), zinc and/ or a vitamin (optionally vitamin D, D2 (or ergocalciferol), D3 (or cholecalciferol) or calcifediol, C, E, B12, B6);
(gg) an anti-androgen drug, optionally bicalutamide, optionally CASODEX™, and optionally the anti-androgen, optionally bicalutamide is administered together with or in combination with ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin) or doramectin (optionally DECTOMAX™),
(hh) a hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
(ii) an alpha-ketoamide (α-ketoamide), wherein optionally the alpha-ketoamide is a structure as described by Zhang et al, J Med. Chem. 2020, 63, 9, 4562-4578, or Meng et al Chem. Sci. (2019) vol. 10, pg 5156 (optionally the structure KAM-2), and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™); an antibiotic (optionally azithromycin or a tetracycline class drug, and optionally the tetracycline class drug comprises doxycycline); chloroquine (or ARALEN™), chloroquine phosphate, chloroquine diphosphate and/or hydroxychloroquine (optionally, PLAQUENIL™); zinc; remdesivir (optionally, GS-5734™, Gilead Sciences); oseltamivir (or TAMIFLU™); and/or, hydrocrtisone; or, any combination thereof;
(jj) a compound, drug or formulation that decreases stomach acid production or decreases stomach pH, wherein optionally the compound, drug or formulation comprises famotidine, or PEPCID™ and optionally the famotidine is administered at a dosage of between about 10 to 60 mg per day, or between about 20 to 40 mg per day, and optionally the famotidine is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), and/or a tetracycline class drug, and optionally the tetracycline class drug comprises doxycycline, or DORYX™, DOXYHEXA™, DOXYLIN™
(kk) a dendrimer, optionally astodrimer sodium (Starpharma, Melbourne, Australia);
(ll) an antihistamine class drug such as azelastine, or ASTELIN™, OPTIVAR™, ALLERGODIL™, brompheniramine, fexofenadine or ALLEGRA™, pheniramine or AVIL™, or chlorpheniramine;
(mm) a selective serotonin reuptake inhibitor (SSRI) class drug, optionally fluvoxamine, or LUVOX™, FAVERIN™, FLUVOXIN™; and/or
(nn) a nicotinic antagonist, a dopamine agonist or a noncompetitive N-Methyl-d-aspartic acid or N-Methyl-d-aspartate (NMDA) antagonist, wherein optionally the nicotinic antagonist, dopamine agonist or noncompetitive NMDA antagonist is 1-adamantylamine or amantadine, or GOCOVRI™, SYMADINE™, SYMMETREL™, optionally administered or dosaged at between about 50 mg to 150 mg, or about 100 mg, per day for a period of between about 7 and 21 days, or about 14 days, and optionally also administered or formulated with an antibiotic (optionally azithromycin or doxycycline), ivermectin, hydroxychloroquine (optionally, PLAQUENIL™) and/or zinc (optionally zinc sulfate, optionally at (50 mg daily), and optionally the amantadine is formulated or administered at 100 mg per day for the first two days of treatment, which optionally can then be elevated to 100 mg twice daily, optionally for the next 10 days;
(oo) an immunosuppressive drug, wherein optionally the immunosuppressive drug comprises tocilizumab or atlizumab, or ACTEMRA™, or ROACTEMRA™, or a calcineurin inhibitor (CNI), wherein the CNI comprises ciclosporin (or cyclosporine or cyclosporin), or NEORAL™, or SANDIMMUNE™, or tacrolimus, or PROTOPIC™, or PROGRAF™, and optionally the immunosuppressive drug is also administered or formulated with an antibiotic (optionally azithromycin or doxycycline), ivermectin, hydroxychloroquine ( optionally, PLAQUENIL™) and/or zinc (optionally zinc sulfate, optionally at (50 mg daily),
and optionally the calcineurin inhibitor (CNI), wherein the CNI comprises ciclosporin (or cyclosporine or cyclosporin) is formulated combination of CNI (optionally cyclosporine) at a dose of 3 mg/kg (180 mg daily) together with 12 mg ivermectin once, and optionally also plus zinc 50 mg base and doxycycline 100 mg bid, optionally all for 10 days;
(pp) a protein kinase inhibitor, wherein optionally the protein kinase inhibitor is a p38 mitogen-activated protein kinase inhibitor, or ralimetinib, and optionally the protein kinase inhibitor is also administered or formulated with an antibiotic (optionally azithromycin or doxycycline), ivermectin, hydroxychloroquine (optionally, PLAQUENIL™) and/or zinc (optionally zinc sulfate, optionally at (50 mg daily);
(qq) an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2 /TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc (optionally zinc sulfate, optionally at (50 mg daily),
and optionally opaganib, or YELIVA™, or opaganib, or YELIVA™ administered or formulated together with an oral and/or inhaled or aerosol chloroquine (or ARALEN™), chloroquine phosphate, chloroquine diphosphate and/or hydroxy chloroquine (optionally, PLAQUENIL™),
and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc (optionally zinc sulfate, optionally at (50 mg daily);
(rr) any combination of (a) to (qq),
wherein optionally any one or several or all of (a) to (rr) with (or formulated with or formulated as an) inhaled formulation and/or formulated, vith or formulated as an oral, intramuscular (IM) or intravenous (IV) formulation, wherein optionally both the inhaled and the oral, IV and/or IM formulations are administered simultaneously or sequentially,
and optionally the inhaled formulation comprises chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) administered simultaneously or overlapping;
and optionally any one or several or all of (a) to (rr), or any therapeutic combination of drugs or a drug, or a pharmaceutical dosage form as provided herein, are administered orally, intramuscularly, subcutaneously, topically, by use of an enema, intravaginally, or intravenously, or administration is by subcutaneous administration, sublingual administration, inhalation or by aerosol (optionally by inhalation of a liquid, an aerosol, a spray, a mist or a powder), by absorbable patch, by use of an implant, or by use of an enema or a suppository.

16. The product of manufacture of claim 15, wherein: the anti-viral drug or medication, or anti-microbial drug, is or comprises: molnupiravir, efavirenz (optionally, SUSTIVA™), tenofovir (optionally tenofovir alafenamide or tenofovir disoproxil or VIREAD™), emtricitabine and tenofovir, nevirapine (or the combination efavirenz or molnupirnvir with emtricitabine and tenofovir, or ATRIPLA™), amprenavir (optionally, AGENERASE™), nelfinavir (optionally, VIRACEPT™) and/or remdesivir (optionally, GS-5734™, Gilead Sciences), a viral RNA-dependent RNA polymerase inhibitor, optionally galidesivir,
and optionally the anti-viral drug or medication is or comprises an anti-retroviral drug or drug combination, and optionally the anti-retroviral drug or drug combination comprises: darunavir and cobicistat (optionally, REZOLSTA™ or PREZCOBIX™); atazanavir and cobicistat (or EVOTAZ™); abacavir, lamivudine and dolutegravir (TRIUMEQ™); tenofovir (or disoproxil or emtricitabine) and elvitegravir and cobicistat (optionally, STRIBILD™); tenofovir (or disoproxil or emtricitabine) and elvitegravir and cobicistat (COMPLERA™ or EVIPLERA™); molnupiravir, efavirenz, emtricitabine and tenofovir (ATRIPLA); lamivudine, nevirapine and stavudine (optionally, TRIOMUNE™); atazanavir and cobicistat (optionally, EVOTAZ™); lamivudine and raltegravir (optionally, DUTREBIS™); lamivudine and dolutegravir (or DOVATO™); doravirine, lamivudine and tenofovir (optionally, DELSTRIGO™); or lamivudine, zidovudine and nevirapine (optionally, CUOVIR-N™);
and optionally the additional anti-viral drug or medication, or anti-microbial drug, is formulated with the chloroquine (optionally, ARALEN™), chloroquine phosphate, chloroquine diphosphate, hydroxychloroquine (optionally, PLAQUENIL™), lopinavir, ritonavir and/or oseltamivir or is formulated separately from the chloroquine (optionally, ARALEN™), chloroquine phosphate, chloroquine diphosphate, hydroxychloroquine (optionally, PLAQUENIL™), lopinavir, ritonavir and/or oseltamivir,
and optionally the anti-viral drug or medication, or anti-microbial drug, or palliative agent comprises: magnesium (Mg, optionally administer intravenously (IV) to maintain a blood concentration of between about 2.0 and 2.4 mmol/L); zinc (optionally a zinc sulphate, acetate, gluconate or picolinate, optionally administered at about 75 to 100 mg/day); at least one vitamin, wherein optionally the at least one vitamin comprises vitamin D (optionally D2 (or ergocalciferol), or Vitamin D3 (or cholecalciferol), optionally administered at about 1000 to 4000 ugm/day), vitamin B12, vitamin B6 (or pyridoxine), vitamin K, vitamin E, and/or vitamin C (optionally administered at 500 mg bid); atorvastatin, or LIPITOR™, SORTIS™ (optionally administered at between about 40 mg/day to 80 mg/day); a flavonoid, plant flavonol or quercetin optionally administered at between about 250 to 500 mg bid; or, melatonin, or CIRCADIN™, SLENYTO™ (optionally between about 6 to 12 mg a day, optionally, at night), and optionally any or all of which are given enterally or parenterally.

17. The therapeutic combination of drugs or drug, pharmaceutical dosage form, drug delivery device, or product of manufacture of claim 1 or claim 2, wherein-(a)
(a) the chloroquine (optionally, ARALEN™), chloroquine phosphate, chloroquine diphosphate, hydroxychloroquine (optionally, PLAQUENIL™), lopinavir, ritonavir and/or oseltamivir are formulated separately or together, or the lopinavir and ritonavir are formulated together and the oseltamivir is formulated separately; and/or
(b) the therapeutic combination of drugs or drug, pharmaceutical dosage form, or the chloroquine (optionally, ARALEN™), chloroquine phosphate, chloroquine diphosphate, hydroxychloroquine (optionally, PLAQUENIL™), lopinavir, ritonavir and/or oseltamivir and/or the anti-viral drug or medication, or anti-microbial drug are formulated or contained in a liquid formulation (optionally sterile saline or water), a spray, a powder, an aerosol, a mist, or any formulation for inhalation,
and optionally the therapeutic combination of drugs or drug, pharmaceutical dosage form, or the chloroquine (optionally, ARALEN™), chloroquine phosphate, chloroquine diphosphate, hydroxychloroquine (optionally, PLAQUENIL™), lopinavir, ritonavir and/or oseltamivir and/or the anti-viral drug or medication, or anti-microbial drug, are coated on the surface of or contained in: a bead, a powder, a particle, or a multilayered bead or particle.

18. A method for inactivating a microbe comprising operating or installing a product of manufacture of claim 1, wherein optionally the microbe comprises a bacteria or a virus, and optionally the virus comprises a respiratory virus, and optionally the virus comprises an influenza virus, a respiratory syncytial virus, a parainfluenza virus, an adenovirus, a rhinovirus, a human metapneumovirus, a hantavirus, an enterovirus, a coronavirus (and optionally the coronavirus comprises a SARS, MERS or a COVID-19 infection causing virus), or a virus in the subfamily Orthocoronavirinae, or a virus in the family Coronaviridae, or a virus in the order Nidovirales.
